(19) United States
(12) Patent Application Publication
(10) Pub. No.: US 2014/0107432 A1
(43) Pub. Date: Apr. 17, 2014

(54) SYSTEM AND METHOD FOR URODYNAMIC EVALUATION

(71) Applicant: THD Inc., Wrentham, MA (US)

(72) Inventors: Filippo BASTIA, SOLIERA (IT); Giancarlo VIGNOLI, BOLOGNA (IT)

(73) Assignee: THD Inc., Wrentham, MA (US)

(21) Appl. No.: 14/046,722

(22) Filed: Oct. 4, 2013

Related U.S. Application Data
(60) Provisional application No. 61/709,613, filed on Oct. 4, 2012.

(51) Int. Cl. A61B 5/20

(52) U.S. Cl. CPC A61B 5/202
USPC 600/301; 600/300

(57) ABSTRACT

A system for urodynamic-evaluation is described. The system has a first reading device configured for gathering selection parameters of a patient, exam parameters, control parameters, and clinical parameters. The system has a processing unit, coupled to the first reading device, configured for determining signals representing an urodynamic-evaluation. The unit has a determination module, a calculation module and a verification module. The system also has a user interface configured for outputting the signals representing the urodynamic-evaluation including at least a predefined evaluation diagram, and values representing the urodynamic evaluation. A method for urodynamic-evaluation for evaluating urodynamic parameters is further described.

### CROSS REFERENCE TO RELATED APPLICATIONS

The present application claims priority to U.S. Prov. App. No. 61/709,613 filed on Oct. 4, 2012, the contents of which are incorporated herein by reference in their entirety.

### FIELD OF APPLICATION

The present disclosure concerns a system for urodynamic-evaluation.

The present disclosure also concerns a method for urodynamic evaluation. Specifically, embodiments of the disclosure provide an urodynamic-evaluation and a tool for understanding an urodynamic tracing and the description provided herein below refers to this field of application with the sole aim of simplifying the description.

### PRIOR ART

Urodynamic is a diagnostic that estimates pressure in a human body, specifically in proximity of bladder or rectum, for evaluating urinary incontinence in women, and prostatic obstruction in men.

It is known that urodynamic provides values that require to be interpreted to understand the real urodynamic health status of a patient.

Urologists are often not able to correctly and completely understand the values provided.

The operator performing the urodynamic examination needs a doctor's help to interpret the urodynamic tracing; as a matter of fact, the values provided are susceptible of wrong interpretation.

From clinical tests, 50% of diagnosis in urodynamic matter are considered not to be accurate.

### SUMMARY

Embodiments of the present disclosure are directed at overcoming the above mentioned drawbacks.

In particular, a system and a method are provided that guarantee accuracy in urodynamic evaluation.

Embodiments of the present disclosure also provide a system and a method providing urodynamic evaluation in an easy way.

Additionally, the present disclosure further provides a system and a method providing urodynamic evaluation that allow any operator to understand urodynamic tracing.

Embodiments of the disclosure also provide a system and a method that give and indication on further examinations required to have a deeper urodynamic evaluation.

In particular, a system for urodynamic-evaluation is described, comprising a first reading device configured for gathering:
Selection parameters (PS1) of a patient (U) representing age of the patient (PS11) and sex of the patient (PS12);
exam parameters (PE1, PEaux) representing at least one type of exam (TE1, TEaux) to which submit the patient (U) as a function of the selection parameters (PS1);
control parameters (PC1, PCaux) representing the presence of clinical parameters for a first type of exam (TE1) chosen for the patient (U);
clinical parameters (P1, P1aux) representing clinical data concerning at least clinical data referred to at least the chosen first type of exam (TE1).

The system further comprises a processing unit (10), coupled to the first reading device (1), and configured for determining signals (Si) representing the urodynamic-evaluation, wherein the unit comprises:
a determination module (101) configured for determining at least a predefined evaluation diagram (DD1, DDaux) representing at least an urodynamic-evaluation (D1, Daux) for at least a type of exam (TE1,TEaux) as a function of the clinical parameters (P1, P1aux);
a calculation module (201) configured for calculating values (VD1, VDaux) representing the at least an urodynamic evaluation (D1, Daux) as a function of at least the predefined evaluation diagram (DD1), transmitting the signals (Si) representing the urodynamic-evaluation to a user interface (11)
a verification module (301) configured for verifying the presence of auxiliary control parameters (PCaux) representing auxiliary clinical parameters (P1aux) representing a presence of auxiliary clinical parameters (P1aux) for a type of an auxiliary exam (TEaux), and in case of presence of the auxiliary control parameters (PCaux), transmitting the auxiliary clinical parameters (P1aux) to the determination module (101).

The system furthermore comprises a user interface (11) configured for outputting the signals (Si) representing the urodynamic-evaluation comprising at least:
the at least a predefined evaluation diagram (DD1, DDaux);
the values (VD1, VDaux) representing the at least an urodynamic evaluation (D1, Daux).

Preferably, the first reading device (1) is configured for further gathering selection parameters (PS1) representing neurologic problems (PS13) in a patient (U), and the processing unit (10) further comprises an evaluation module (200) configured for managing the urodynamic-evaluation for the patient (U) with neurologic problems and transmitting the signals (Si) representing the urodynamic-evaluation to a user interface (11), wherein the processing unit furthermore comprises a second verification module (104) configured for verifying if the selection parameters (PS13) is representing presence of neurologic problems, and in the case it is, transmitting a first response signal (RS1) to the evaluation module (200), otherwise, transmitting a second response signal (RS2) to the determination module (101) for managing the non-neurologic case.

Preferably the first reading device (1) comprise a visual interface. Alternatively, the first reading device (1) comprises a network interface.

The present disclosure also describes a method for urodynamic evaluation, for evaluating urodynamic parameters, comprising the steps of
gathering selection parameters (PS1) of a patient (U) representing age of the patient (PS11) and sex of the patient (PS12);
gathering exam parameters (PE1, PEaux) representing at least one type of exam (TE1, TEaux) (es. pressure flow) to which submit the patient (U) as a function of the selection parameters (PS1);
gathering control parameters (PC1, PCaux) representing the presence of clinical parameters for the a first type of exam (TE1) chosen for the patient (U);
gathering clinical parameters (P1, P1aux) representing clinical data concerning at least clinical data referred to at least the chosen first type of exam (TE1);
providing a processing unit (10), configured for determining signals (Si) representing the urodynamic-evaluation, wherein the unit id configured for:
   determining at least a predefined evaluation diagram (DD1, DDaux) representing at least an urodynamic-evaluation (D1, Daux) for at least a type of exam (TE1,TEaux) as a function of the clinical parameters (P1, P1aux);
   calculating values (VD1, VDaux) representing of the at least an urodynamic evaluation (D1, Daux) as a function of at least the predefined evaluation diagram (DD1);
   transmitting the signals (Si) representing the urodynamic-evaluation to a user interface (11);
   verifying the presence of auxiliary control parameters (PCaux) representing of auxiliary clinical parameters (P1aux) representing of a presence of auxiliary clinical parameters (P1aux) for a type of an auxiliary exam (TEaux);
   in case of presence of the auxiliary control parameters (PCaux), transmitting the auxiliary clinical parameters (P1aux) to the determination module (101);
providing a user interface (11) configured for outputting the signals (Si) comprising the at least a predefined evaluation diagram (DD1, DDaux) and the values (VD1, VDaux) representing of the at least an urodynamic evaluation (D1, Daux), the signals (Si) representing the urodynamic-evaluation.

Preferably the method further comprises the steps of:
gathering selection parameters (PS1) representing neurologic problems (PS13) in a patient (U);
verifying if the selection parameters (PS13) is representing presence of neurologic problems;
in the case it is, transmitting a first response signal (RS1) to the evaluation module (200), configured for managing the urodynamic-evaluation for the patient (U) with neurologic problems, and for transmitting the signals (Si) representing the urodynamic-evaluation to the user interface (11);
otherwise, transmitting a second response signal (RS2) to the determination module (101) for managing the non-neurologic case.

Preferably the method further comprise the steps of providing a visual interface for gathering parameters and/or for displaying the signals (Si) representing the urodynamic-evaluation.

Preferably the method further comprise the steps of providing a network interface for gathering parameters and/or for displaying the signals (Si) representing the urodynamic-evaluation.

According to the several embodiments of the present disclosure, the system and method for urodynamic-evaluation, as described, can achieve the following technical effects:
guaranteeing accuracy in urodynamic evaluation;
providing urodynamic evaluation in an easy way;
giving an indication on further examinations required to have a deeper urodynamic evaluation.

The technical effects cited and additional technical effects of the disclosure will appear in a more detailed form in the description, given herein below, of an embodiment provided by way of approximate, non-limiting example with reference to the accompanying drawings.

### BRIEF DESCRIPTION OF THE FIGURES

FIG. 1a shows a block diagram of the system according to the disclosure.

FIG. 1b show in detail a portion of the block diagram of FIG. 1a.

FIGS. 2 to 12 show flow diagrams of the operation of the system of the present disclosure mainly in non-neurologic urodynamic-evaluation.

FIGS. 3a, 3b, 3c, 3d and 3e show examples of monograms of urodynamic-evaluation in non-neurologic patients.

FIGS. 13 to 16 show flow diagrams of the operation of the system of the present disclosure in neurologic urodynamic-evaluation.

FIGS. 14a, 14b and 14c shows examples of monograms of urodynamic-evaluation in neurologic patients.

### DETAILED DESCRIPTION OF PREFERRED EMBODIMENTS

A system for urodynamic-evaluation according to embodiments of the present disclosure comprises a first reading device configured for gathering selection parameters of a patient, exam parameters, control parameters, clinical parameters, wherein the system comprises a processing unit, coupled to said first reading device, configured for determining signals representing an urodynamic-evaluation, the unit comprising a determination module, a calculation module and a verification module, and wherein the system comprises a user interface configured for outputting the signals representing the urodynamic-evaluation comprising at least a predefined evaluation diagram, and values representing the urodynamic evaluation.

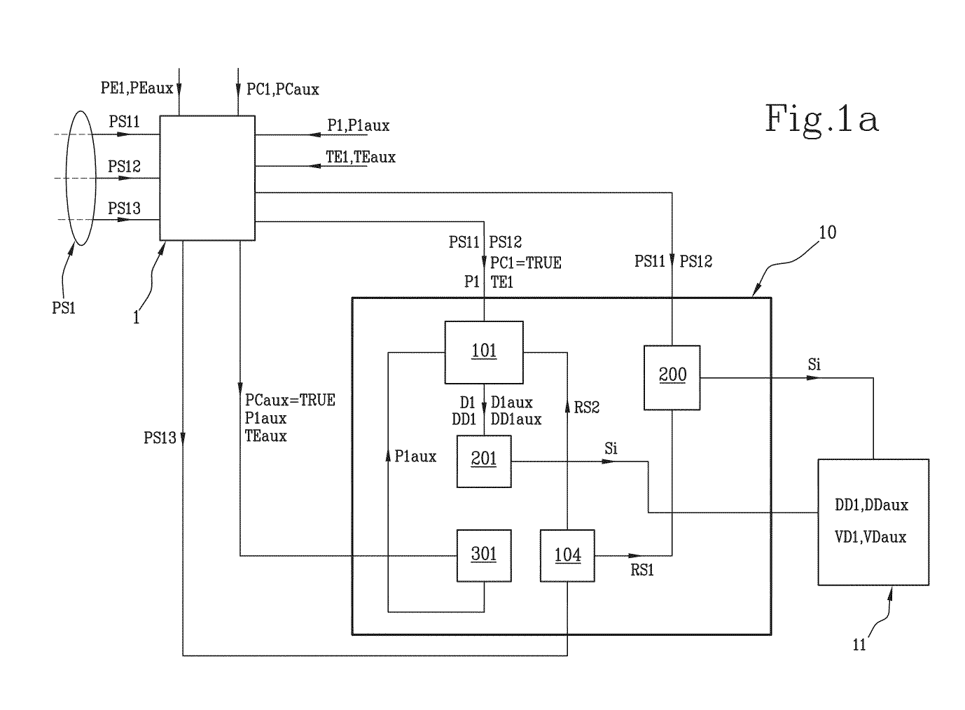

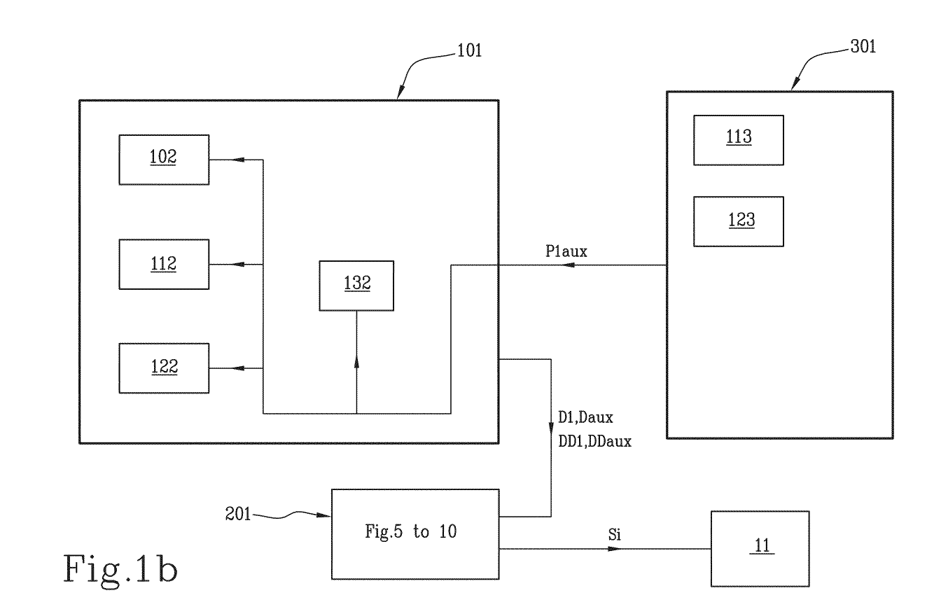

The system for urodynamic-evaluation according to embodiment of the disclosure is shown in its more general implementation in FIG. 1.

In such a Figure, the system comprises a first reading device 1. Preferably, the first reading device 1 comprises a visual interface. Preferably, the first reading device 1 comprises a network interface.

The first reading device 1 is configured for gathering selection parameters PS1 of a patient U.

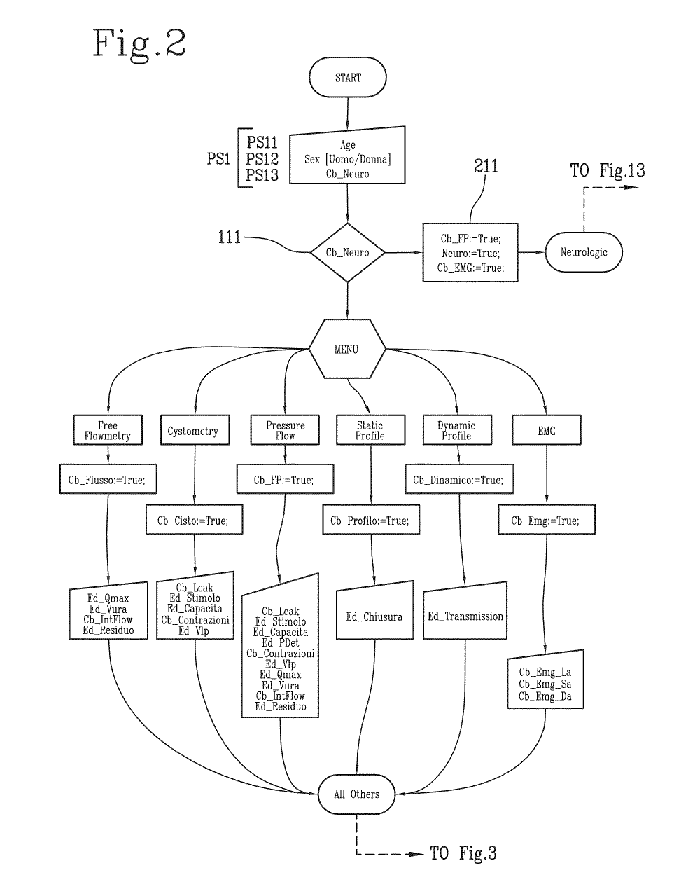

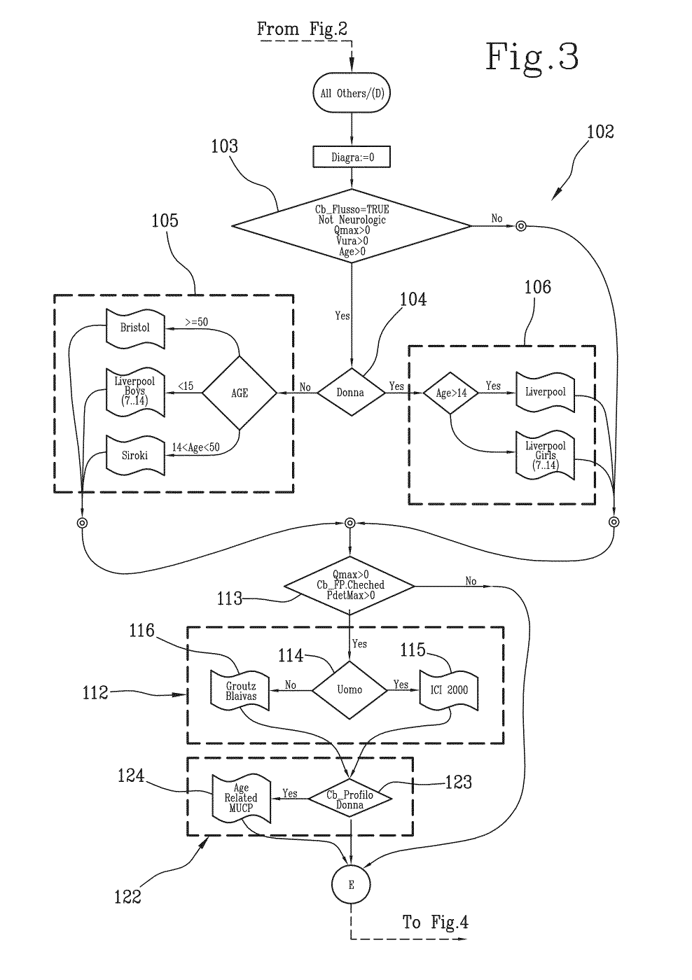

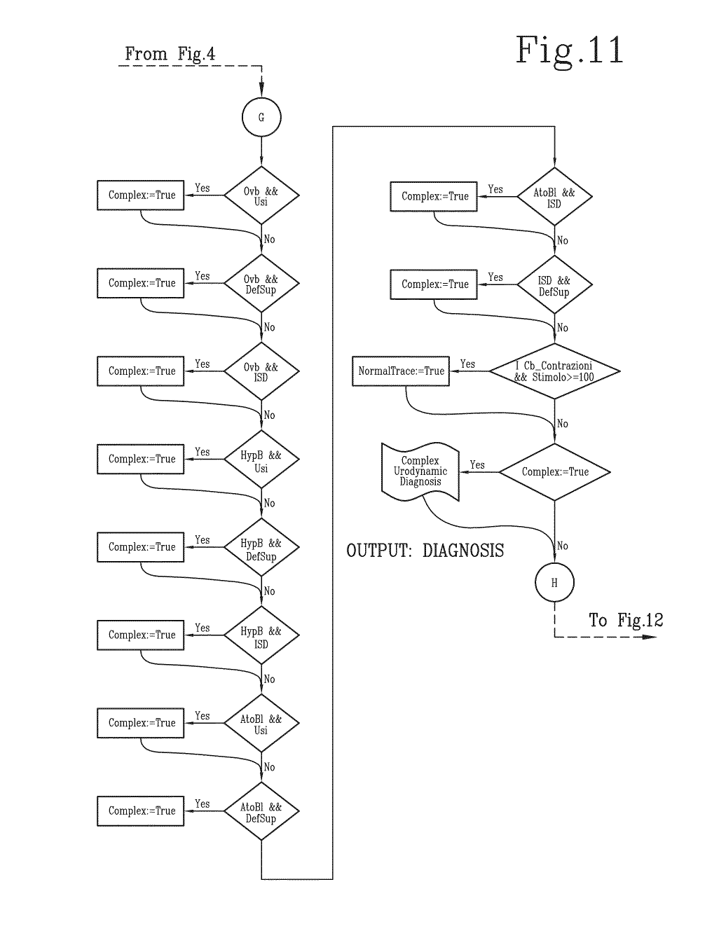

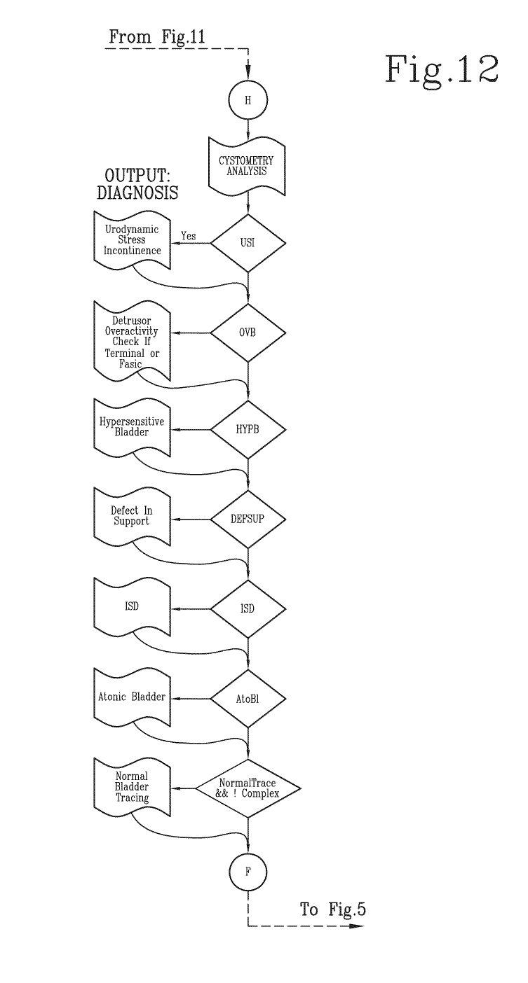

In a first embodiment, the selection parameters PS1 of the patient U represent age of the patient PS11 and sex of the patient PS12. In this embodiment described, specifically, with reference to FIGS. 2-12 the case is shown wherein parameters of a patient U without neurologic problems are evaluated.

In an alternative embodiment the case is shown wherein parameters of a patient U with neurologic problems are evaluated.

In this case, in addition to the age PS11 and the sex PS12, the selection parameter PS1 of the patient U will represent also neurologic problems PS13. An operating module 111 is configured for checking the value true or false of the parameter PS13.

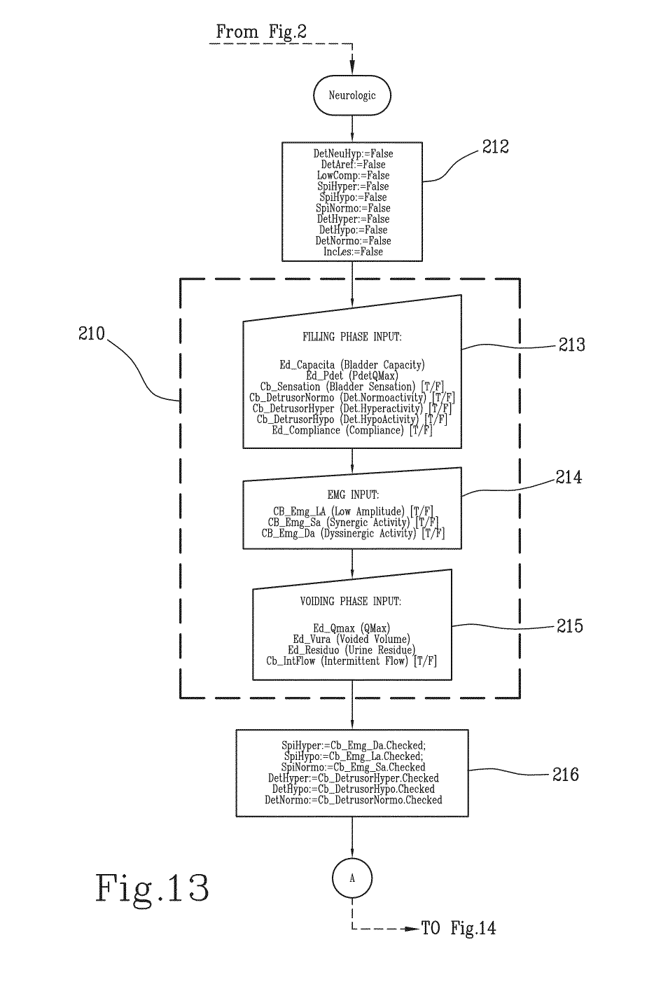

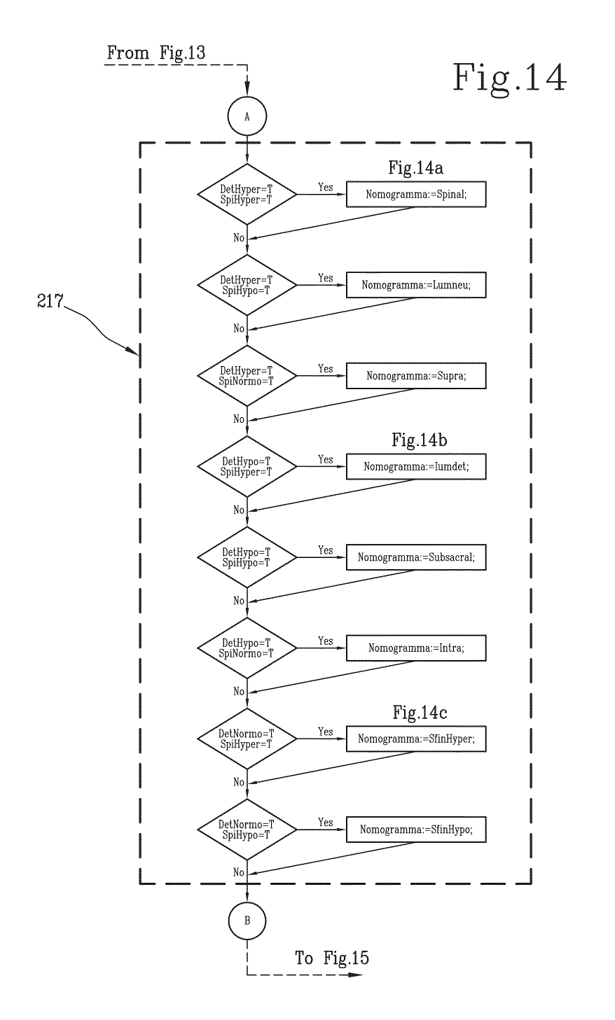

This embodiment will be better explained hereinafter in the description and mainly with reference to FIGS. 13-16 of the present application.

According to the first embodiment, the first reading device 1 is configured for gathering exam parameters PE1, PEaux representing at least one type of exam TE1, TEaux to which submit the patient U.

According to the present disclosure, exam parameters PE1, Peaux are defined as a function of the selection parameters PS1.

According to the disclosure, the urologist selects the correct exam TE1, TEaux more suitable for a patient U, then selects the exam parameters PE1, Peaux representing at least one type of exam (TE1, TEaux) to which submit the patient (U) as a function of the selection parameters (PS1).

Preferably, type of exams TE1 selectable are one or more in the list of Free Flowmetry, Cystometry, Pressure Flow, Static Profile, Dynamic Profile, EMG.

The first type of exam can be one among the above mentioned exams, as shown below.

### 1. Flowmetry Exam

Preferably, with reference to FIG. 2, clinical parameters P1, P1aux comprise for Flowmetry exam, at least:
Ed_Qmax=maximum flow rate;
Ed_Vura=voided volume;
Cb_IntFlow=intermittent flow;
Ed_Residuo=Residual urine;

The system according to the disclosure deals with features of flowmetry as in the following.

### 1.1 Male Flowmetry

Voided volume should be at least 150 ml and preferably 200 ml. For voided volume lower than 150 ml (correspondingly less in children: 50 to 100 ml) a warning indicate the voiding pattern has to be interpreted with caution for possible erroneous result due to inadequate voided volume and suggest to repeat the test.

Since males show a significant decline in flow rate with age, the system considers:
Siroky nomogram for men under 55, preferably under 50;
Bristol nomogram for men over 55, preferably over 50.

In adults the Free Flowmetry predictive value is also reported, in order to reduce the need of pressure/flow study: if the Qmax is below 10 ml/s, the chance of the patient to have a bladder outlet obstruction is 90%; if the Qmax is 10 ml/s to 15 ml/s the incidence of obstruction falls to 71%; if the Qmax is over 15 ml/s the chance of obstruction is 50% (high pressure/high flow system).

### 1.2 Female Flowmetry

Unlikely male, female do not show statistically significant variations in urine flow rate with respect to age, parity or first versus repeated voiding.

The 10th centile of the Liverpool Nomogram for the maximum urine flow rate has been considered to be the most useful discriminant for a final urodynamic diagnosis of voiding difficulties in females.

### 1.3 Uroflowmetry in Pediatric Age

Urine flowmetry together with ultrasound assessment of residual urine is by far the most common procedure in pediatric urodynamic practice. The results of the examination decide whether the child requires an invasive urodynamic investigation. Recently nomograms in centile forms have been reported both for girls and boys under 14 years of age for a wide range of voided volume. These nomograms have been utilized in the system for automated analysis of flow in children.

### 1.4 Residual Urine

Threshold values delineating what constitutes an abnormal PVR (PostVoid Residual) are in short defined. The system take into account only large PVRs (>200 ml) with a warning indicating that values greater than 200 ml may be associated with an increased risk of urinary retention, upper urinary tract dilation and renal insufficiency.

### 2. Cystometry Exam

Preferably, with general reference to FIG. 2, clinical parameters P1, P1aux comprise for Cystometry exam at least:
Cb_Leak=Urinary Leakage;
Ed_Stimolo=Stimolous;
Ed_Capacità=bladder capacity;
Cb_Contrazioni=contractions;
Ed_Vlp=Lowest Vesical Pressure

The system of the disclosure deals with features of Cystometry exam as in the following.

Bladder storage function should be assessed in terms of bladder sensation, detrusor activity, bladder compliance and bladder capacity.

Furthermore the urethra should be assessed in term of competency through cough (urodynamic stress incontinence) and strain (Valsalva leak point pressure). The failure to store urine during the filling phase may be either a result of an abnormal (overactive or oversensitive) detrusor or an abnormal (i.e. too weak) sphincter complex. In mixed incontinence the two situations coexist.

The system of the disclosure takes into account:
FSF (first sensation of filling)
Cystometric capacity
involuntary detrusor contractions spontaneous or on provocation
Compliance, expressed as increase in bladder volume per centimetre of water increase in pressure (ml/cm H20). In the normal bladder the change in pressure from empty to full should be less than 10 cm H20 giving a Figure for normal compliance of greater than 40 ml/cm H20
Urine Leakage through the external meatus during cough
VLPP at 200 ml of filling.

Detrusor Idiopatic overactivity (overactive bladder) is diagnosed in presence of significant detrusor overactivity, subjectively observed by the examiner.

There has been considerable confusion over the objective definition of DO (Detrusor overactivity) with some investigators labelling patients as having DO if there is an increase of pdet (Detrusor Pressure) greater than 15 cm H20 during filling.

However, the ICS standardization document of 1988 made it clear that D0 is characterised by phasic contractions (pressure rise and fall) without specifying a minimum change in pdet. Waves of an amplitude of less than 5 cm H20 are difficult to detect using most modern urodynamic equipments (35).

However, it is undoubtedly true that low-pressure DO waves (5 cm H20-15 cm H20) can produce troublesome symptoms of urgency particularly in women.

Bladder hypersensitivity is diagnosed in presence of an early first sensation of filling and an early first sensation to void (usually <100 ml) which persist into normal and strong desire without concomitant phasic detrusor contractions.

Reduced Compliance is diagnosed when the pressure at cystometric capacity is greater than 10 cm H20.

Urodynamic stress incontinence is diagnosed when urine leak from the external meatus is observed when the patient raises her intrabdominal pressure in the absence of a detrusor contraction.

IDS is diagnosed when VLPP at 200 ml of filling is lower than 60 cm H2O.

### 3. Pressure Flow Exam

Preferably, with general reference to FIG. 2, clinical parameters P1, P1aux comprise for Pressure Flow exam at least:
Cb_Leak=Urine Leakage;
Ed_Stimolo=Stimolous;
Ed_Capacità=bladder capacity;
Cb_Contrazioni=contractions;
Pdet=Detrusor Pressure
Ed_Vlp=Lowest Vesical Pressure
Ed_Qmax=maximum flow rate;
ED_Vura=voided volume;
Cb_IntFlow=intermittent flow;
Ed_Residuo=Residual urine.

The system of the disclosure deals with features of Pressure Flow exam as in the following.

Conventional urodynamics is able to provide information on both filling and voiding phases of micturition cycle.

This is achieved by measuring bladder and abdominal pressure with real-time computational determination of detrusor pressure by using the formula pdet=pm−pabd

The accurate measurement of pdet is entirely dependent on the accuracy with wich pabd (interabdominal pressure) and pves (Intravesical pressure) are measured.

The 2001 K35 report says that after derivation, pdet is 0 cm H20 to 6 cm H20 in 80% of cases.

As previously said, before starting the pressure/flow study, quality control is ensured by a warning that ask the examiner to check that pdet is under 6 cm H20.

The pressure-flow relation is much better defined in men than in women, in male patients the diagnosis of BOO is made by plotting the maximum flow rate (Qmax) against detrusor pressure at Qmax (pdet Qmax) into the iCS nomogram.

BOO is also calculated without reference to nomogram utilizing the equation:

BOOI(Bladder Outlet Obstruction index)=pdetQmax−2Qmax

If the B001 is greater than 40 then BOO exist; if it is below 40 then no definite BOO exists. Under 20 patient is unobstructed.

In addition the system analyses the detrusor contractility utilizing the equation:

BCI(Bladder Contraction Index)=pdetQmax−5Qmax

A BCI of greater than 150 suggests strong contractility, whereas less than 100 is poor. BCI 100-150 is the normal range.

The definitions and nomograms that are used to describe BOO in men do not apply to women, since men and women have unique micturitional characteristics.

Voiding phase of female patient is analysed through the Groutz-Blaivas nomogram. nomogram includes 4 zones: unobstruction, mild, moderate, severe obstruction. BOO is defined as free Qmax<12 ml/s combined with pdet Qmax>20 cm H20.

### 4. Static or Dynamic Profile Exam

Preferably, with general reference to FIG. 2, clinical parameters P1, P1aux comprise for Dynamic Profile exam at least:
Transmission.

Preferably, clinical parameters P1, P1aux comprise for EMG (Electromyography) exam:
EMG_La=Low amplitude EMG
EMG_Sa=Sphincter activity
EMG_Da=Dyssinergic activity

### 4.1 Male Profilometry.

Male profilometry has limited clinical relevance as a diagnostic tool for bladder outlet obstruction, because it doesn't reflect the dynamic behaviour of the urethra during micturition. Conversely, it may be used to evaluate the degree of sphincter lesion after radical prostatectomy and to follow spontaneous recovery. Several papers reports the multifactorial origin of incontinence after radical prostatectomy There are controversies about the assessment of sphincteric function after radical prostatectomy. Although VLPP (vesical leak point pressures) has not been shown to correlate with severity of incontinence, MUCP (Maximum Urethral Closure Pressure) appears to be a more useful measurement in the post-RP population, since in incontinent patients has been reported significantly lower than in continent patients.

Basing mostly on personal experience, the system of the disclosure describes three incontinent sub-groups:

1. MUCP between 60 and 80 cm H20 suggesting a mild sphincteric weakness,

2. MUCP between 40 and 60 cm H20 suggesting a moderate sphincteric weakness

3. MUCP under 40 cm H20 suggesting a severe sphincteric weakness.

### 4.2 Urethral Profilometry and Dysfunctional Voiding

The ICS has defined Dysfunctional voiding as an intermittent and/or fluctuating flow rate due to involuntary intermittent contractions of peri-urethral striated muscle during voiding in neurologically normal patients.

In male the pattern, that has been called also “pseudodyssinergia” seems to account for 35% of bladder outlet obstruction especially in young adults.

In female dysfunctional voiding is quite common in painful bladder and related pelvic floor syndromes.

Obviously, the “gold standard” for diagnosing the disorder is the pressure/flow study with EMG.

It is not infrequent, however, to observe in office practice the performance of flowmetry followed by cystometry and urethral profilometry.

In presence of an interrupted free flow and with an MUCP exceeding 10 cm H20 the normal age-dependent MUCP

value in the female and a fixed value of 120 cmH20 in male, a pressure/flow study with EMG is warranted. The latter usually evidentiate a poor relaxing sphincter with mild- to moderate obstruction in female and equivocal obstruction with underactive detrusor in male.

### 5. Neurogenic Bladder.

Urodynamic diagnosis in neurogenic bladder follows special features (terminology, sensation, compliance) in a condition similar to ECG in the patient with a pacemaker.

As previously said, the system provides a specific algorithm for neurogenic patient.

Neurogenic bladder dysfunction may be due to:

1. dysfunction of the detrusor;
2. dysfunctions of the sphincter;
3. a combination of both.

When one suspect neurogenic bladder a pressure/flow study with EMG become mandatory. Two types of information can be obtained from EMG: a simple indication of muscle behaviour, the so-called kinesiological EMG, or an electrical correlation of muscle pathology.

During urodynamic investigation a kinesiological EMG is usually obtained.

Sphincter activity may be: synergic, dyssinergic or non-relaxing and low-amplitude.

Synergic activity indicates a progressive increase of EMG activity during filling of the bladder (guarding reflex) followed by a timely relaxation of the pelvic floor during voiding. Dyssinergic or non-relaxing activity indicates an increase of EMG activity during voiding (sometimes the activity may result unmodified or “waxing and waining”)

Low amplitude EMG indicates a reduced electrical activity both during filling and voiding phase.

The finding may indicate a peripheral denervation of the muscle for which a neurophysiological approach, through a needle EMG and oscilloscope, is recommended in the final report.

Correspondingly, detrusor function may be: normo-, hyper-, hypo-active.

The patterns of detrusor-sphincter function reported in the boxes of the display identifies eight types of neurourodynamic diagnoses, according to Madersbacher;
supra-pontine reflex bladder (detrusor hyperactivity-synergic EMG activity);
spinal reflex bladder (detrusor hyper activity—dyssinergic or non-relaxing EMG activity);
sub-sacral lesion (detrusor hypoactivity—low amplitude EMG);
lumbosacral lesion I (detrusor hyperactivity—low amplitude EMG);
lumbosacral lesion II (detrusor hypoactivity—dyssinergic or non-relaxing EMG activity);
intra-pelvic lesion I (detrusor hypoactivity—synergic EMG activity);
intra-pelvic lesion II (detrusor normoactivty—dyssinergic or non-relaxing EMG activity);
intra-pelvic lesion III (detrusor normoactivity—low amplitude EMG activity).

In addition to neurourodynamic diagnosis, voiding phase is analysed through A-G nomogram in male and Groutz-Blaivas nomogram in female to evaluate the presence or absence of a mechanical obstruction.

The absence or presence of sensation during filling account for a complete versus incomplete neurogenic lesion.

### System Operation

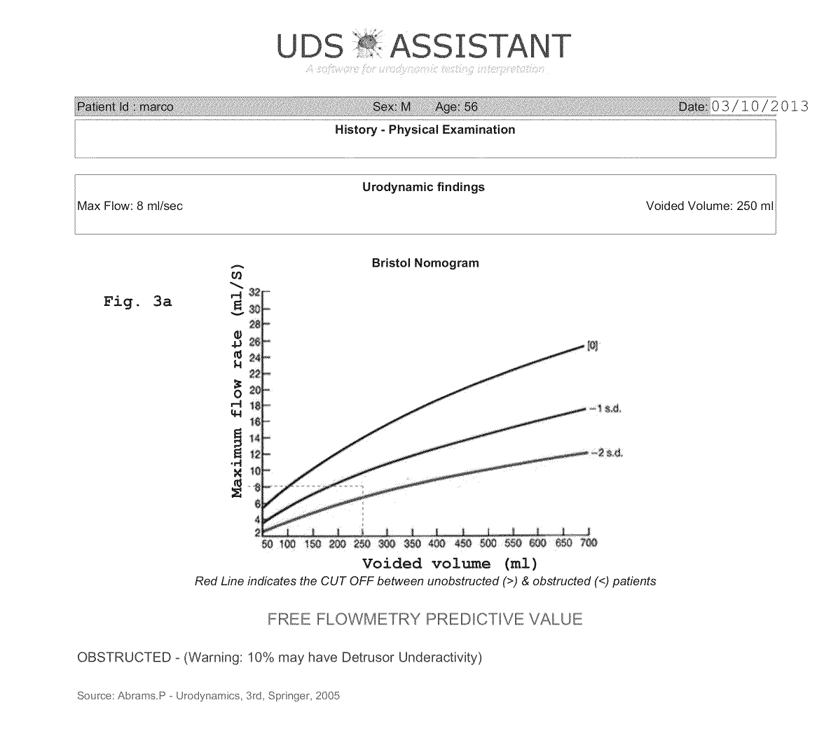

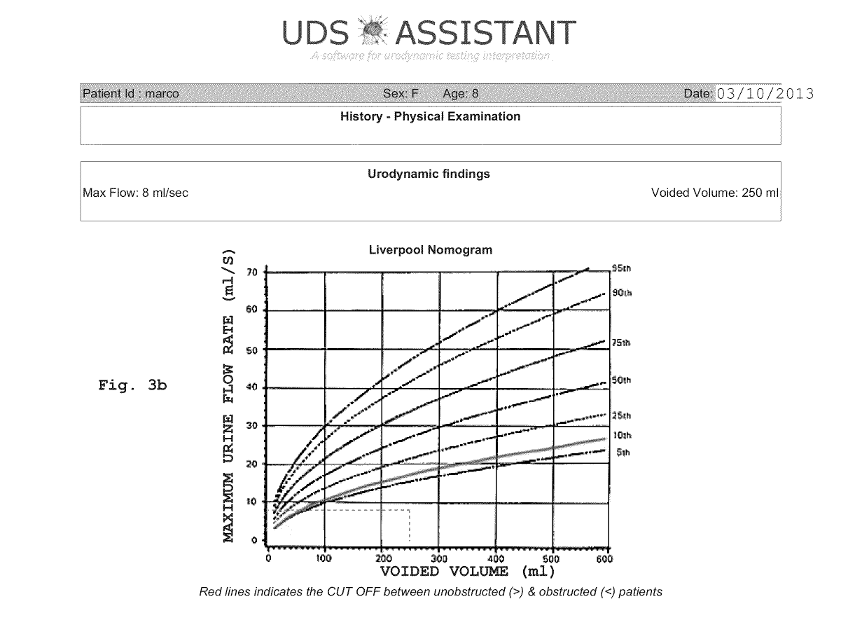

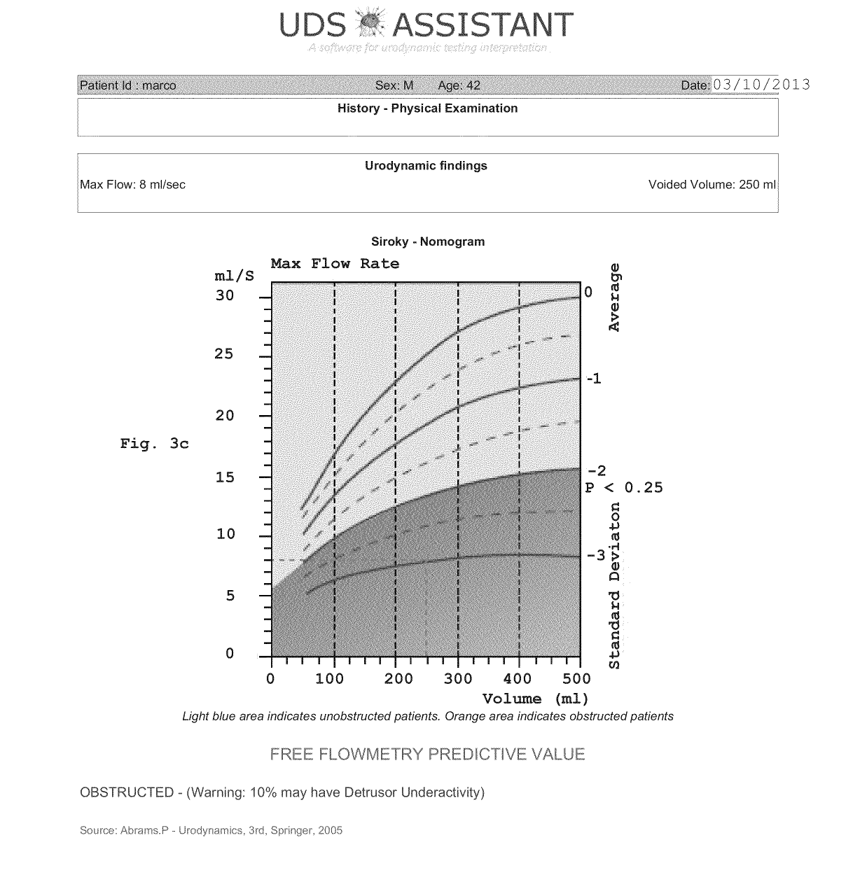

Operation of the system will now be described with specific reference to FIGS. 3-16.

When the urologist selects an exam TE1 for starting the evaluation, the system checks for the presence of a specific parameter, namely a control parameter (PC1, PCaux) representing the presence of a clinical parameter P1 for the type of exam TE1 chosen for the patient U.

To this aim, the first reading device 1 is configured for gathering control parameters (PC1, PCaux) representing the presence of clinical parameters for the first type of exam TE1 chosen for the patient U, and indicating that it is possible to carry out the exam.

Preferably, control parameters PC1, PCaux selectable comprises cb_flusso for the Flowmetry exam, cb_cisto for Cystometry exam, cb_FP for pressure flow exam, cb_profilo for Static Profile exam, cb_dinamico for Dynamic Profile exam, cb_emg for EMG exam.

As far as the presence of control parameter has been checked, the system starts the evaluation as a function clinical parameter (P1, P1aux) specific for the selected exam.

Specifically, the clinical parameters are numerical values derived from the respective exam.

To this aim, the first reading device 1 is configured for gathering clinical parameters P1 representing clinical data concerning at least clinical data referred the chosen first type of exam TE1.

As depicted in FIG. 1, advantageously, according to embodiments of the disclosure, the system for urodynamic-evaluation comprises a processing unit 10, coupled to the first reading device 1, and configured for determining signals Si representing an urodynamic-evaluation.

Generally it is to note that in the present context and in the following claims, the processing unit 10 is depicted as subdivided in different functional modules (memory modules or operating modules) to the only purpose to clearly and completely describe the functions.

Actually such a processing unit 10 may consist of just one electronic device, suitably programmed to implement the described functions, and the different modules may correspond to hardware entities and/or software routines forming the programmed device. Alternatively or in addition, said functions may be performed by a plurality of electronic devices on which the above mentioned functional modules may be arranged.

The processing unit 10 may avail itself of one or more processors for the carrying out of the instructions contained in the memory modules.

The above mentioned functional modules may be further distributed on different either local or remote computers in accordance with the architecture of the network in which they reside.

Advantageously, the processing unit 10 comprises a determination module 101 configured for determining at least a predefined evaluation diagram (DD1, DDaux) representing at least an urodynamic-evaluation (D1, Daux) for at least a first type of exam (TE1,TEaux) as a function of the clinical parameters (P1, P1aux).

With reference to FIG. 3, in an embodiment of the disclosure, the urologist selects flowmetry as type of first exam TE1 and Qmax and Vura as clinical parameters P1.

According to the disclosure, the system is configured for dynamically evaluating the patient U condition as a function of the above mentioned clinical parameters.

Specifically, with reference to FIG. 3, the determination module 101 comprises a flowmetry unit (102) configured for carrying out a flowmetry analysis.

The flowmetry unit (102) comprises a first checking module (103) configured to check the presence of the control parameter PC1, namely the presence of flux (Cb_flusso=TRUE in FIG. 3) for the Flowmetry Exam.

The checking module 103 is further configured to check compliance of:
selection parameters PS1, namely Age >0 and not neurologic,
clinical parameters P1, namely Qmax>0 and Vura>0, with the Exam being performed.

The flowmetry unit (102) further comprises a second checking module 104 configured to evaluate the sex (selection parameter S12) of the patient U.

If the sex is male, the second checking module 104 activates a first diagram module 105 configured to show a predefined evaluation diagram (DD1) representing at least an urodynamic-evaluation (D1) for at least a first type of exam (TE1) as a function of the clinical parameters (P1).

Specifically, the first diagram module 105 is configured to show:
a Bristol evaluation diagram if the age of the patient is more or equal to 50 years;
a Liverpool Boys evaluation diagram if the age of the male patient is less than 15 years;
a Siroki evaluation diagram if the age of the patient is less than 50 years.

Examples are shown in FIGS. 3a, 3b and 3c respectively with reference to Bristol evaluation diagram (age=56), Liverpool Boys evaluation diagram (age=13) and Siroki evaluation diagram (age=42), in all diagram being considered Qmax=8 ml/s and Vura=250 ml.

If the sex is female, the second checking module 104 activates a second diagram module 106 configured to show a predefined evaluation diagram (DD1) representing at least an urodynamic-evaluation (D1) for at least a first type of exam (TE1) as a function of the clinical parameters (P1).

Specifically, the second diagram module 106 is configured to show:
a Liverpool evaluation diagram if the age of the female patient is more than 14 years;
a Liverpool girls evaluation diagram if the age of the female patient is less than 14 years.

The system, according to the disclosure comprises a verification module 301 configured for verifying the presence of auxiliary control parameters (PCaux) representing auxiliary clinical parameters (P1aux) representing a presence of auxiliary clinical parameters (P1aux) for a type of auxiliary exam (TEaux).

The verification module 301 is configured, in case of presence of the auxiliary control parameters (PCaux), for transmitting the auxiliary clinical parameters (P1aux) to the determination module (101).

Specifically, with reference to FIG. 3, the determination module 101 is coupled to the verification module 301.

The determination module 101 is configured for receiving the auxiliary clinical parameters (P1aux) in order to determine a predefined evaluation diagram DD1, DDaux representing at least an urodynamic-evaluation (D1, Daux) for at least a first type of exam (TE1) and at least an auxiliary exam TEaux as a function of the clinical parameters (P1, P1aux).

The determination module 101 further comprises a Pressure flow unit (112) configured for carrying out a Pressure flow analysis, namely an analysis based on an auxiliary type of exam TEaux.

The Pressure flow unit (112) is coupled to the verification module 301.

The verification module 301 comprises a third checking module (113) configured to check the presence of an auxiliary control parameter (PCaux) for the auxiliary type of Exam(TEaux).

In an embodiment of the disclosure, the auxiliary type of Exam comprises the Pressure Flow exam and the control parameter PCaux comprises the pressure flow cb_FP.

The third checking module (113) is further configured to check compliance of selection parameters (PS1) and clinical parameters (P1aux) with the Exam being performed.

Preferably, clinical parameters P1aux comprises
Cb_Leak=Urine Leakage;
Ed_Stimolo=Stimolous;
Ed_Capacità=bladder capacity;
Cb_Contrazioni=contractions;
Pdet=Detrusor Pressure
Ed_Vlp=Lowest Vesical Pressure
Ed_Qmax=maximum flow rate;
ED_Vura=voided volume;
Cb_IntFlow=intermittent flow;
Ed_Residuo=Residual urine;

In an embodiment of the disclosure, as shown in FIG. 3, the Exam being performed is the Pressure Flow exam and the clinical parameter (P1aux) is PdetMax>0.

The pressure flow unit (112) further comprises a fourth checking module 114 configured to evaluate the sex (selection parameter S12) of the patient U.

If the sex is male, the fourth checking module 104 activates a third diagram module 115 configured to show a predefined evaluation diagram (DDaux) representing an auxiliary urodynamic-evaluation (Daux) for at least a first type of exam (TEaux) as a function of the clinical parameters (P1aux).

Specifically, the fourth diagram module 115 is configured to show a ICI 2000 evaluation diagram.

An example is shown in FIG. 3d with Qmax=8 ml/s, Vura=250 ml and Pdetmax=10.

If the sex is female, the fourth checking module 114 activates a fourth diagram module 116 configured to show a predefined evaluation diagram (DDaux) representing an auxiliary urodynamic-evaluation (Daux) for at least a first type of exam (TEaux) as a function of the clinical parameters (P1aux). Specifically, the fourth diagram module 116 is configured to show a Blaivas evaluation diagram.

An example is shown in FIG. 3e with Qmax=8 ml/s, Vura=250 ml and Pdetmax=10.

The verification module 301 comprises a sixth checking module (134) configured to check the presence of an auxiliary control parameter (PCaux) for the auxiliary type of Exam(TEaux).

According to the disclosure, the determination module (101) further comprises a Static Profile unit (122) configured for carrying out a Static profile analysis. auxiliary type of exam TEaux.

The Static Profile unit (122) is coupled to the verification module 301. The verification module 301 comprises a fifth checking module (123) configured to check the presence of an auxiliary control parameter (PCaux) for the auxiliary type of Exam(TEaux).

In an embodiment of the disclosure, the auxiliary type of Exam comprises the Static Profile exam and the control parameter PCaux comprises the profile cb_profilo.

The fifth checking module (123) is further configured to check compliance of selection parameters (PS1) and clinical parameters (P1aux) with the Exam being performed.

In an embodiment of the disclosure, the Exam being performed is the Pressure Flow exam and the clinical parameter (P1aux) is PdetMax>0.

The fifth checking module (123) is configured to evaluate the sex (selection parameter S12) of the patient U.

The fifth checking module 123 is configured for activating a fifth diagram module 124 configured to show a Age related MUCP (Maximum urethral closure pressure) diagram, if the selection parameter Ps, namely the sex is representative of a female.

Conversely, the fifth checking module 123 is configured for activating a sixth checking module 133 to check the presence of further auxiliary control parameters (PCaux) for further auxiliary exams (TEaux), if the selection parameter Ps, namely the sex, is representative of male.

According to the disclosure, the determination module (101) further comprises a Cystometry unit (132) configured for carrying out a Cystometry analysis.

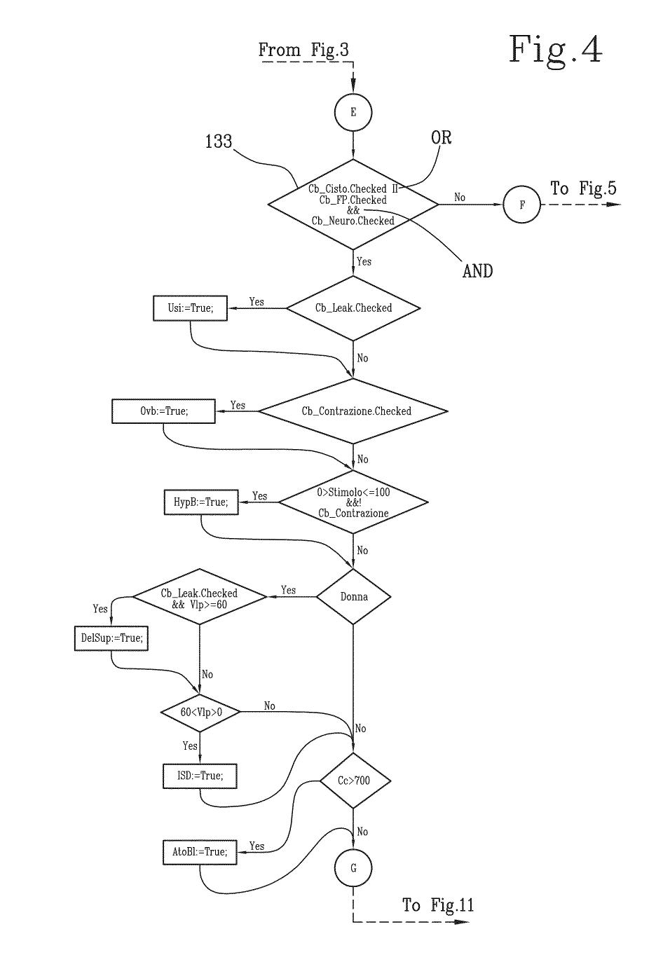

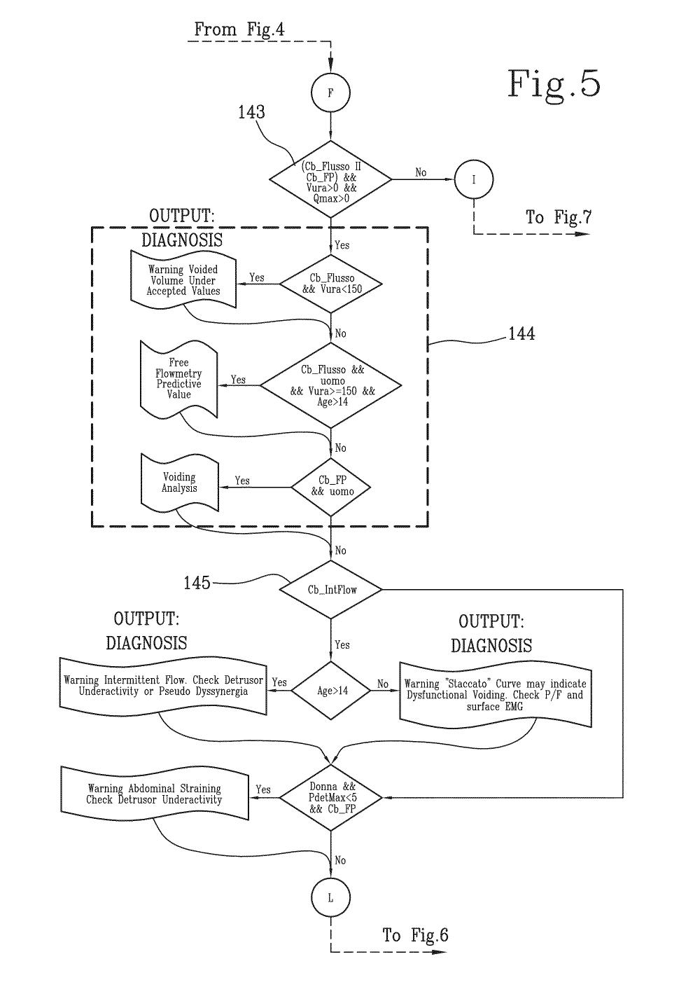

In an embodiment of the disclosure, as shown in FIG. 4, the Exam being performed is the Cystometry exam and the clinical parameter (P1aux) are Cb_Leak=Urine Leakage and Cb_Contrazioni=contractions.

With reference to FIGS. 4, 11 and 12, output diagnosis are clearly evaluated as a function of the cystometry and Pressure flow clinical parameters (stimolo and contrazione; see also paragraph 2 and 3 of the present application and FIG. 4).

The Figures are considered self-explaining also with reference to paragraph 2 and 3 of the present application.

With reference to FIG. 1, the processing unit 10, according to the disclosure, comprises a calculation module (201) configured for calculating values (VD1, VDaux) representing the at least an urodynamic evaluation (D1, Daux) as a function of at least the predefined evaluation diagram (DD1).

Otherwise stated, the calculation module 201 provides also an urodynamic evaluation in terms of written diagnosis of the problem encountered if the values calculated are not in compliance with standard values of the reference parameters used in the system.

The written analysis is complementary to the diagram analysis previously described and is reproduced in the bottom part of the example screenshots.

Specifically, the calculation module 201 comprises a first values check module (133) (FIG. 4) configured to check the presence of control parameter for Cystometry OR Pressure Flow Exam and for the condition of neurologic.

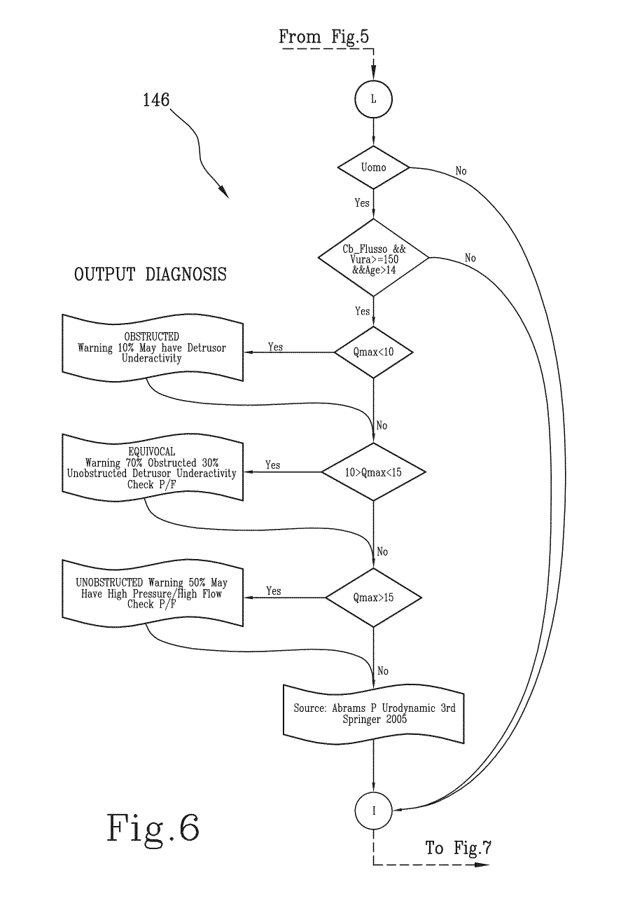

If the check is FALSE, the calculation module 201 is configured for activating a second values check module (143) (FIG. 5).

With specific reference to FIG. 5, the second values check module (143) is configured to check the presence of control parameter for Flowmetry OR Pressure Flow Exam, and to check values Vura>0 and Qmax>0.

The steps of generating of the urodynamic evaluation as a function of different input values will now be described.

The calculation module comprises a third values check module (144) configured for checking the presence of the control parameter Cb_Flusso and a value Vura<150.

If the check is TRUE, then the system provides a Warning of Voided Volume Under Accepted Values. Else if the check is TRUE and the selection parameter PS12 is male and Vura>=150 and age>14, then system provides a Free Flowmetry Predictive Value.

The third values check module (144) is configured for checking the presence of the control parameter Cb_FP and if the selection parameter PS12 is male; if the check is TRUE than the system provides a voiding Analysis value.

The calculation module comprises a forth values check module (145) configured for checking the presence of the control parameter Cb_IntFlow.

If the check is TRUE and age>14 years then system provides a Warning “Staccato” Curve may Indicate Dysfunctional voiding. Check P/F and surface EMG. If the check is TRUE and age <=14 years then system provides a Warning of Intermittent Flow. Check Detrusor Underactivity or Pseudo Dyssynergia. If the check is FALSE, AND the selection parameter PS12 is female and PdetMax<5 and the control parameter Cb_FP is present, then the system provides a Warning of Abdominal Straining and Check Detrusor Underactivity.

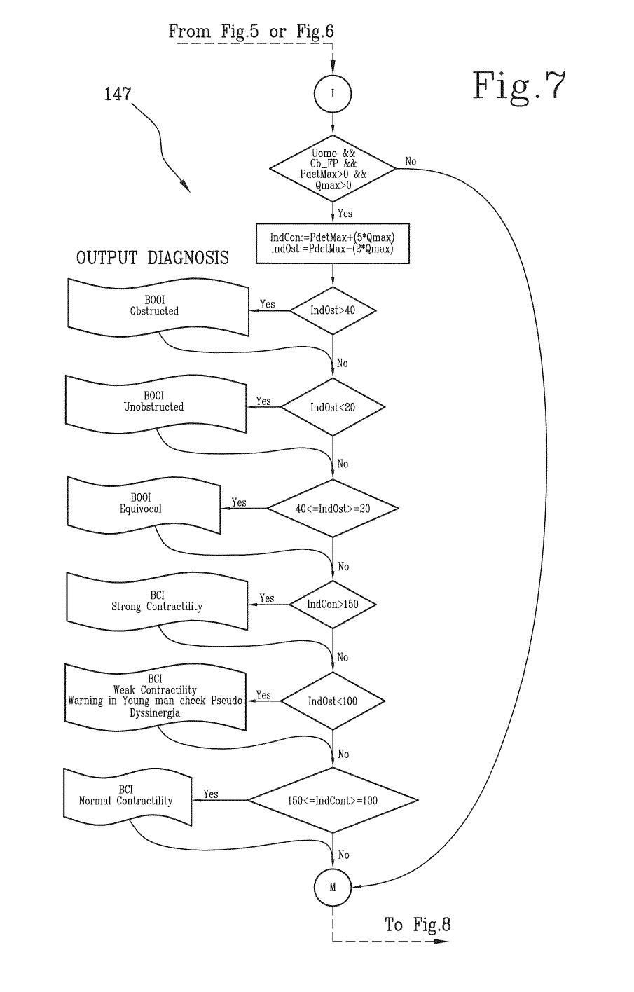

With specific reference to FIG. 6, the calculation module comprises a fifth values check module (146) configured for determining an output diagnosis as a function of the selection parameter PS12=male and the presence of the control parameter Cb_Flusso and Vura>=150 and age<14 years.

If the checks are all TRUE then:

if Qmax<10 then the outpuT is: OBSTRUCTED Warning 10% May have Detrusor Underactivity;

if 10<Qmax<15 then the output is: EQUIVOCAL Warning 70% Obstructed 30% Unobstructed Detrusor Underactivity check P/F;

if Qmax>15 then the output is UNOBSTRUCTED Warning 50% may have High Pressure/High Flow; Check P/F.

With specific reference to FIG. 7, the calculation module comprises a sixth values check module (147) configured for determining an output diagnosis as a function of:
the selection parameter PS12=male,
the presence of the control parameter Cb_FP,
PdetMax>0,
QMax>0,
a calculated value of an Obstruction Index=PdetMax−(2*Qmax),
a calculated value of Contraction Index=PdetMax+(5*Qmax).

With reference to FIG. 7, output diagnosis are clearly evaluated and the Figure is considered self-explaining also with reference to paragraph 3 of the present application.

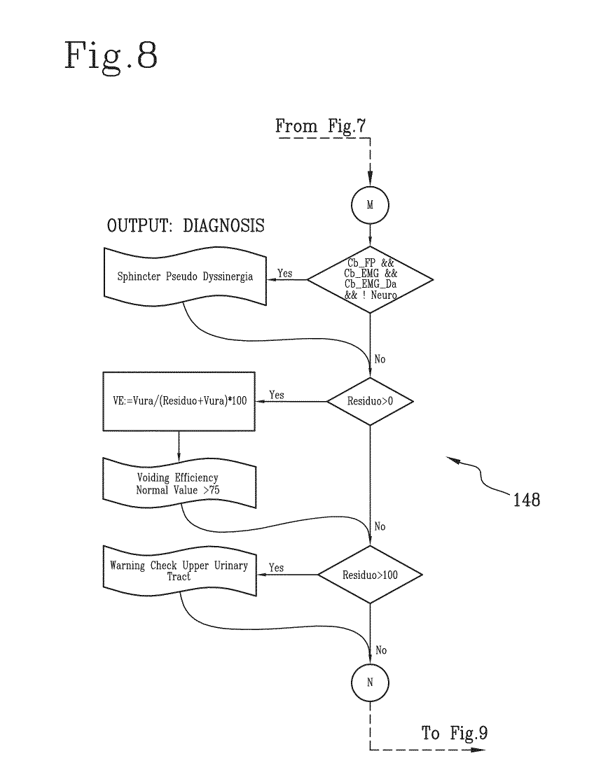

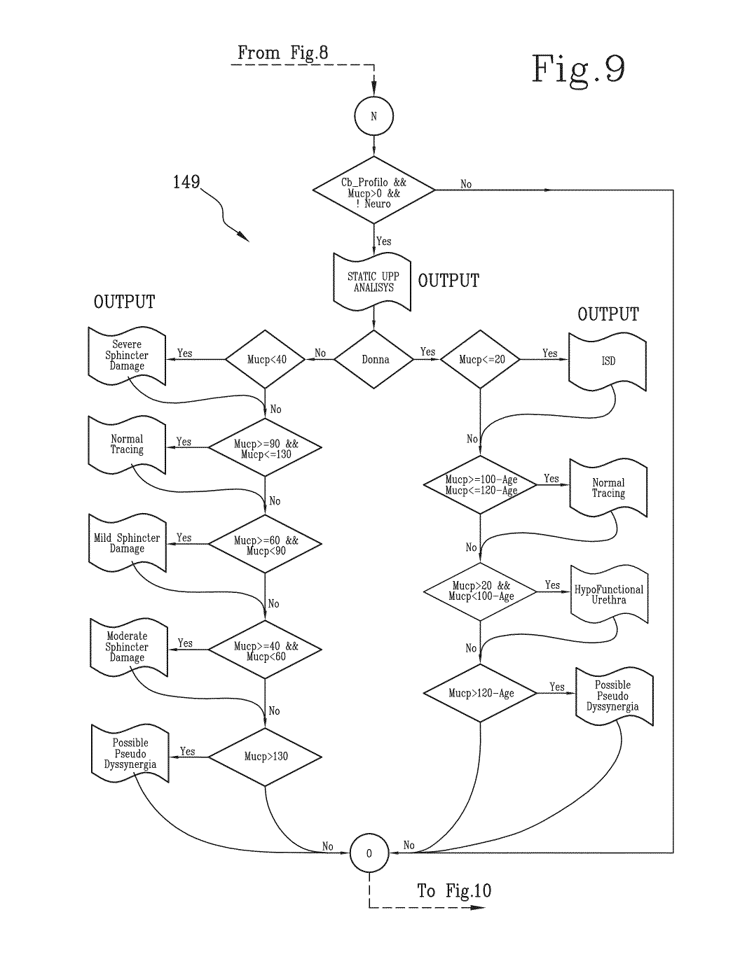

With specific reference to FIG. 8, the calculation module comprises a seventh values check module (148) configured for determining an output diagnosis as a function of the presence of the control parameters Cb_FP AND cb_EMG AND cb_EMG_Da AND neurologic patient.

With reference to FIG. 8, output diagnosis are clearly evaluated as a function of different values of the clinical parameter Residuo.

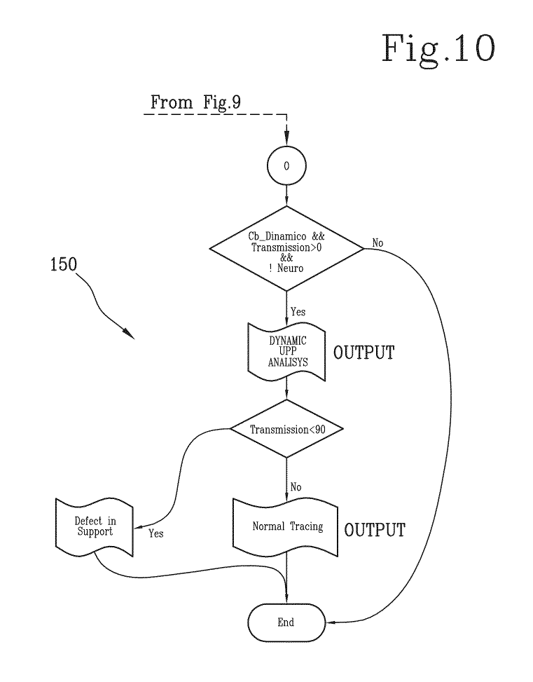

With specific reference to FIG. 9, the calculation module comprises a eighth values check module (149) configured for determining an output diagnosis as a function of the presence of the control parameters Cb_Profile (Static Profilometry) and MUCP>0 (see paragraph 4 of the present application) AND cb_EMG AND cb_EMG_Da AND neurologic patient.

With reference to FIG. 9, output diagnosis are clearly evaluated as a function of the selection parameter PS12 (male or female) and of different values of MUCP (Maximum Urethral Closure Pressure). The Figure is considered self-explaining also with reference to paragraph 4 of the present application.

With specific reference to FIG. 10, the calculation module comprises a ninth values check module (150) configured for determining an output diagnosis as a function of the presence of the control parameters Cb_PDinamico (Dynamic Profilometry) and transmission>0 (see paragraph 4 of the present application) AND neurologic patient.

With reference to FIG. 10, output diagnosis are clearly evaluated as a function of values of the clinical parameter Transmission. The Figure is considered self-explaining also with reference to paragraph 4 of the present application.

Turning now to the general aim of the disclosure, with reference to FIG. 1, the system, specifically the first reading device (1) is configured for gathering selection parameters (PS1) representing neurologic problems (PS13) in a patient (U).

According to the disclosure, in this case the processing unit (10) comprises an evaluation module (200) configured for managing the urodynamic-evaluation for the patient (U) with neurologic problems. The evaluation module (200) is also configured for transmitting signals (Si) representing the urodynamic-evaluation to a user interface (11).

The control unit 10, according to the disclosure further comprises a second verification module (104) configured for verifying if the selection parameters (PS13) is representing presence of neurologic problems. In the case it is, the second verification module (104) is configured for transmitting a first response signal (RS1) to the evaluation module (200), otherwise, it is configured for transmitting a second response signal (RS2) to the determination module (101) for managing the not neurologic case.

Operation of the system in the neurologic case will now be described, with reference also to the previous paragraph (Neurogenic bladder).

In the following, the prefix “Det” is intended a short indication for “Detrusor”, where detrusor functions may be normo, hyper, or hypo active.

The prefix “Spi” is intended as a short indication for Sphincter, where sphincter activity may be synergic, dyssinergic, and low amplitude.

Returning now to FIG. 2, the system according to the disclosure is capable of checking, by means of the operating module 111, whether or not the patient U is a neurologic patient, i.e. a patient with neurologic problems.

According to the disclosure, in this case, the evaluation module (200) is configured for managing the urodynamic-evaluation for the patient (U) with neurologic problems.

The evaluation module 200 comprises a first neuro checking module 211 configured for checking the presence of the control parameter PC1,PCaux namely the presence of pressure flow Cb_Fp, and the presence of electromyography Cb_EMG in a neurologic urodynamic-evaluation.

With reference to FIG. 13, the evaluation module 200 comprises a reset module 212 for resetting values of all the clinical parameters P1, P1aux involved in the exam.

Specifically, the reset module performs the following actions:

DetNeuHyp:=False

DetAref:=False

L0wComp:=Fase

SpiHyper:=False

Spii-iypo:=False

SpiNorrno:=False

DetHyper::False

DetHypo:=False

DetNurrno:=False

lncLes'=False.

The evaluation module 200 comprises an input module 210 configured for managing input of clinical parameters, wherein the module comprises:
a filling phase input module 213 configured for inputting
Ed_Capacita=Bladder Capacity;
Ed_Pdet=PdetQMax;
Cb_Sensation=Bladder Sensation [Y/F]
Cb_DetrusorNormo=Det.Normoactivity [T/F]
Cb_DetrusorHyper=Det.Hyperactivity [T/F]
Cb_DetrusorHypo=Det.Hypoactivity [T/F]
Ed_Compliance=Compliance [T/F]
an EMG input module 214 configured for inputting
CB_Emg_LA=Low Amplitude [T/F]
CB_Emg_Sa=Synergic Activity (T/F]
CB_Emg_Da=Dyssinergic Activity [T/F]
a voiding phase input module 215 configured for inputting:
Ed_Qmax=QMax
Ed_Vura=voided Vulume
Ed_Residuo=Urine Residue
Cb_IntFlow=intermittent Flow [T/F]

The evaluation module 200 comprises a setting module 216 configured for setting values of the of clinical parameters of activity of the sphincter and of the detrusor; specifically:

SpiHype-r:=Cb_Emg_Da.Checked;

SpiHypo:=Cb_Emg_La.Checked;

SpiNormo:=Cb_Emg_Sa.Checked;

DetHyper:=Cb_DetrusorHyper.Checked;

DelHyp0:=Cb_DetrusorHypoChecked;

DetNormo:=Cb_DetrusurN0rmo.Checked.

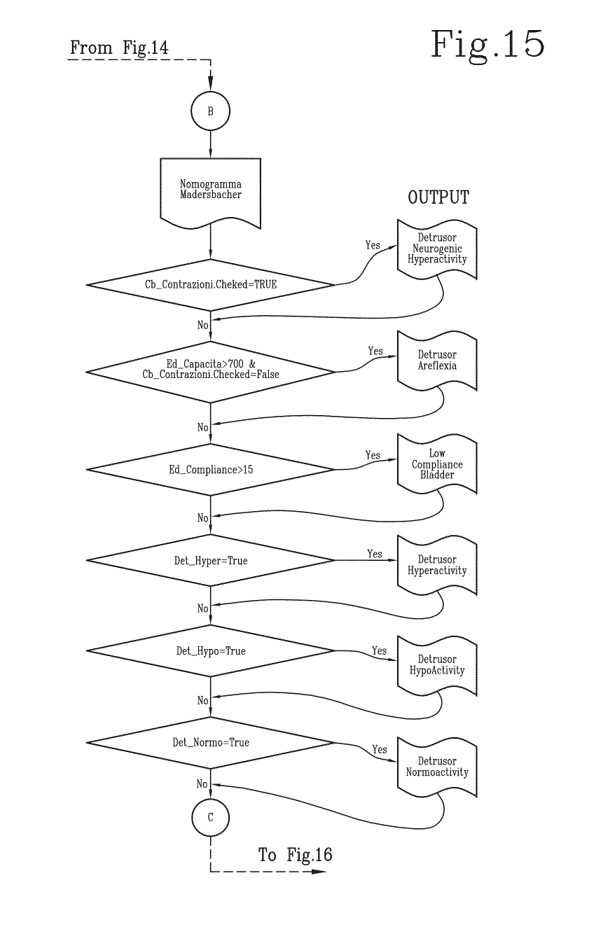

With specific reference to FIG. 14, every combination of sphincter activity with detrusor activity is evaluated by a specific nomogram.

The evaluation module 200 comprises a detrusor-sphincter module 217 configured to associate a specific nomogram to any combination of detrusor/sphincter activity.

Specifically:

If Detrusor Hyperactivity=TRUE and Sphincter Hyperactivity=TRUE then the reference nomogram is a Spinal Reflex Bladder nomogram; en example of such a nomogram is shown in FIG. 14a.

If Detrusor Hyperactivity=TRUE AND Sphincter Hypoactivity=TRUE then the reference nomogram is a Lumbosacral lesion (lumneu) nomogram.

If Detrusor Hyperactivity=TRUE and Sphincter Normoactivity=TRUE then the reference nomogram is a Suprapontine Reflex Bladder nomogram.

If Detrusor Hypoactivity=TRUE and Sphincter Hyperactivity=TRUE then the reference nomogram is a Lumbosacral lesion (lumdet) nomogram; en example of such a nomogram is shown in FIG. 14b.

If Detrusor Hypoactivity=TRUE and Sphincter Hypoactivity=TRUE then the reference nomogram is a Subsacral lesion nomogram.

If Detrusor Hypoactivity=TRUE and Sphincter Normoactivity=TRUE then the reference nomogram is a Intrapelvic Lesion of Bladder innervation nomogram.

If Detrusor Normoactivity=TRUE and Sphincter Hyperactivity=TRUE then the reference nomogram is Sphincter Hyperreflexia only nomogram; en example of such a nomogram is shown in FIG. 14c.

If Detrusor Normoactivity=TRUE and Sphincter Hypoactivity=TRUE then the reference nomogram is a Sphincter Hypo/Areflexia nomogram.

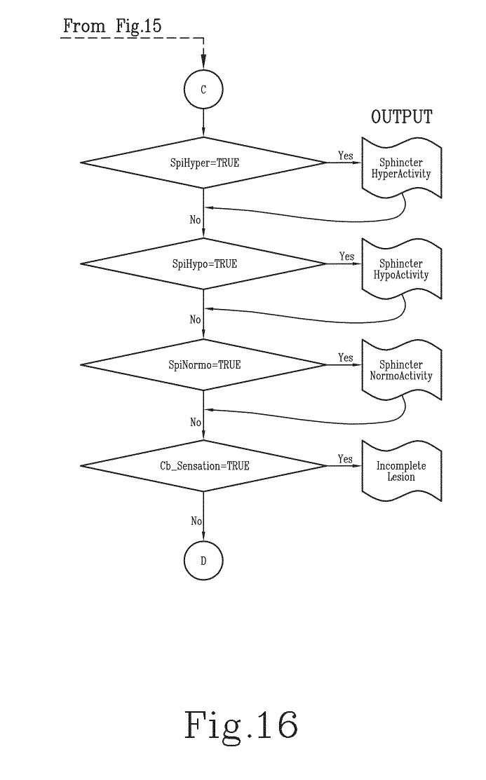

With reference to FIGS. 15 and 16 The evaluation module 200 comprises a Madersbacher module 218 configured to associate a specific diagram to combinations of clinical parameters of urodynamic-evaluation in a neurologic patient.

Specifically, with reference to FIG. 15, the Madersbacher module 218 is configured for evaluating:

if contractions=TRUE then the output is a Detrusor Neurogenic Hyperactivity,

else if the bladder capacity is >700 and contractions=FALSE then output is Detrusor Areflexia,

else if Compliance >15

else if Detrusor Hyperactivity=TRUE then output is Detrusor Hyperactivity,

else if Detrusor Hypoactivity=TRUE then output is Detrusor Hypoactivity,

else if Detrusor Normoactivity=TRUE then output is Detrusor Normoactivity.

With specific reference to FIG. 16,

else if Sphincter Hyperactivity=TRUE then output is Sphincter Hyperactivity,

else if Sphincter Hypoactivity=TRUE then output is Sphincter Hypoactivity,

else if Sphincter Normoactivity=TRUE then output is Sphincter Normoactivity,

else if the control parameter Sensation=TRUE then the output is Incomplete Lesion.

### Method According to the Present Disclosure

The present disclosure also describes a method for urodynamic evaluation, for evaluating urodynamic parameters, comprising the steps of
gathering selection parameters (PS1) of a patient (U) representing age of the patient (PS11) and sex of the patient (PS12);
gathering exam parameters (PE1, PEaux) representing at least one type of exam (TE1, TEaux) (es. pressure flow) to which submit the patient (U) as a function of the selection parameters (PS1);
gathering control parameters (PC1, PCaux) representing the presence of clinical parameters for the a first type of exam (TE1) chosen for the patient (U);
gathering clinical parameters (P1, P1aux) representing clinical data concerning at least clinical data referred to at least the chosen first type of exam (TE1);
providing a processing unit (10), configured for determining signals (Si) representing the urodynamic-evaluation, wherein the unit id configured for:
   determining at least a predefined evaluation diagram (DD1, DDaux) representing at least an urodynamic-evaluation (D1, Daux) for at least a type of exam (TE1,TEaux) as a function of the clinical parameters (P1, P1aux);
   calculating values (VD1, VDaux) representing of the at least an urodynamic evaluation (D1, Daux) as a function of at least the predefined evaluation diagram (DD1);
   transmitting the signals (Si) representing the urodynamic-evaluation to a user interface (11);
   verifying the presence of auxiliary control parameters (PCaux) representing of auxiliary clinical parameters (P1aux) representing of a presence of auxiliary clinical parameters (P1aux) for a type of an auxiliary exam (TEaux);
   in case of presence of the auxiliary control parameters (PCaux), transmitting the auxiliary clinical parameters (P1aux) to the determination module (101);
providing a user interface (11) configured for outputting the signals (Si) comprising the at least a predefined evaluation diagram (DD1, DDaux) and the values (VD1, VDaux) representing of the at least an urodynamic evaluation (D1, Daux), the signals (Si) representing the urodynamic-evaluation.

Preferably the method further comprises the steps of:
gathering selection parameters (PS1) representing neurologic problems (PS13) in a patient (U);
verifying if the selection parameters (PS13) is representing presence of neurologic problems;
in the case it is, transmitting a first response signal (RS1) to the evaluation module (200), configured for managing the urodynamic-evaluation for the patient (U) with neurologic problems, and for transmitting the signals (Si) representing the urodynamic-evaluation to the user interface (11);
otherwise, transmitting a second response signal (RS2) to the determination module (101) for managing the non-neurologic case.

Preferably the method further comprise the steps of providing a visual interface for gathering parameters and/or for displaying the signals (Si) representing the urodynamic-evaluation.

Preferably the method further comprise the steps of providing a network interface for gathering parameters and/or for displaying the signals (Si) representing the urodynamic-evaluation.

1. A system for urodynamic-evaluation comprising:
A first reading device configured for gathering:
Selection parameters of a patient representing:
age of the patient;
sex of the patient;
exam parameters representing at least one type of exam to which submit the patient as a function of the selection parameters;
control parameters representing the presence of clinical parameters for the a first type of exam chosen for the patient;
clinical parameters representing clinical data concerning at least clinical data referred to at least the chosen first type of exam;
a processing unit, coupled to said first reading device, and configured for determining signals representing the urodynamic-evaluation, wherein the unit comprises:
a determination module configured for determining at least a predefined evaluation diagram representing at least an urodynamic-evaluation for at least a type of exam as a function of the clinical parameters;
a calculation module configured for
calculating values representing the at least an urodynamic evaluation as a function of at least the predefined evaluation diagram;
transmitting the signals representing the urodynamic-evaluation to a user interface;
a verification module configured for:
verifying the presence of auxiliary control parameters representing auxiliary clinical parameters representing a presence of auxiliary clinical parameters for a type of an auxiliary exam;
in case of presence of the auxiliary control parameters, transmitting the auxiliary clinical parameters to the determination module;
a user interface configured for outputting the signals representing the urodynamic-evaluation comprising at least:
the at least a predefined evaluation diagram;
the values representing the at least an urodynamic evaluation.

2. The system for urodynamic-evaluation according to claim 1 wherein:
the first reading device is configured for further gathering:
selection parameters representing neurologic problems in a patient;
and the processing unit comprises:
an evaluation module configured for managing the urodynamic-evaluation for the patient with neurologic problems and transmitting the signals representing the urodynamic-evaluation to a user interface;
a second verification module configured for:
verifying if the selection parameters is representing presence of neurologic problems;
in the case it is, transmitting a first response signal to the evaluation module, otherwise, transmitting a second response signal to the determination module for managing the non-neurologic case.

3. The system for urodynamic-evaluation according to claim 1 wherein said first reading device comprise a visual interface.

4. The system for urodynamic-evaluation according to claim 1 wherein said first reading device comprises a network interface.

5. The system for urodynamic-evaluation according to claim 1 wherein the determination module comprises a flowmetry unit configured for carrying out a flowmetry analysis.

6. The system for urodynamic-evaluation according to claim 5 wherein the flowmetry unit comprises:
a first checking module configured to:
check the presence of the control parameter for the flowmetry exam;
check compliance of selection parameters and clinical parameters with the Exam being performed;
a second checking module configured to evaluate the selection parameter of the patient and
if the selection parameter is representative of a male person, the second checking module activates a first diagram module configured to show a first predefined evaluation diagram representing at least an urodynamic-evaluation for the first type of exam as a function of the clinical parameters;
if the selection parameter is representative of a female, the second checking module activates a second diagram module configured to show a second predefined evaluation diagram representing at least an urodynamic-evaluation for the first type of exam as a function of the clinical parameters.

7. The system for urodynamic-evaluation according to claim 6 wherein the first diagram module is configured to show:
a Bristol evaluation diagram if the age of the patient is more or equal to 50 years;
a Liverpool Boys evaluation diagram if the age of the male patient is less than 15 years;
a Siroki evaluation diagram if the age of the patient is less than 50 years;
and the second diagram module is configured to show:
a Liverpool evaluation diagram if the age of the female patient is more than 14 years;
a Liverpool girls evaluation diagram if the age of the female patient is less than 14 years.

8. The system for urodynamic-evaluation according to claim 7 wherein the determination module comprises a Pressure Flow unit configured for carrying out a Pressure Flow analysis.

9. The system for urodynamic-evaluation according to claim 8 wherein the pressure flow unit is coupled to the verification module, wherein the verification module comprises:
a third checking module configured to:
check the presence of an auxiliary control parameter for the Pressure Flow exam;
check compliance of selection parameters and clinical parameters with the Exam being performed.
a fourth checking module configured to evaluate the selection parameter of the patient and
if the sex is male, the fourth checking module activates a third diagram module configured to show a predefined evaluation diagram representing an auxiliary urodynamic-evaluation for at least a first type of exam as a function of the clinical parameters;
if the sex is female, the fourth checking module activates a fourth diagram module configured to show a predefined evaluation diagram representing an auxiliary urodynamic-evaluation for at least a first type of exam as a function of the clinical parameters.

10. The system for urodynamic-evaluation according to claim 9 wherein the third diagram module is configured to show an ICI 2000 evaluation diagram and the fourth diagram module is configured to show a Groutz Blaivas evaluation diagram.

11. The system for urodynamic-evaluation according to claim 10 wherein the determination module comprises a static profile unit configured for carrying out a static profile analysis.

12. The system for urodynamic-evaluation according to claim 10 wherein the static profile unit is coupled to the verification module, wherein the verification module comprises:
a fifth checking module configured to
check the presence of an auxiliary control parameter for the Static Profile exam;
if the sex is female, the fifth checking module activates a fifth diagram module configured to show an age related diagram
if the sex is male, the fourth checking module activates a sixth checking module to check the presence of further auxiliary control parameters for further auxiliary exams.

13. A method for evaluating urodynamic parameters comprising the steps of:
gathering selection parameters of a patient representing:
age of the patient;
sex of the patient;
gathering exam parameters representing at least one type of exam to which submit the patient as a function of the selection parameters;
gathering control parameters representing the presence of clinical parameters for the a first type of exam chosen for the patient;
gathering clinical parameters representing clinical data concerning at least clinical data referred to at least the chosen first type of exam;
providing a processing unit, configured for determining signals representing the urodynamic-evaluation, wherein the unit id configured for:
determining at least a predefined evaluation diagram representing at least an urodynamic-evaluation for at least a type of exam as a function of the clinical parameters;
calculating values representing of the at least an urodynamic evaluation as a function of at least the predefined evaluation diagram;
transmitting the signals representing the urodynamic-evaluation to a user interface;
verifying the presence of auxiliary control parameters representing of auxiliary clinical parameters representing of a presence of auxiliary clinical parameters for a type of an auxiliary exam;
in case of presence of the auxiliary control parameters, transmitting the auxiliary clinical parameters to the determination module;
providing a user interface configured for outputting the signals comprising the at least a predefined evaluation diagram, and the values representing of the at least an urodynamic evaluation, the signals representing the urodynamic-evaluation.

14. The method according to claim 13 comprising the steps of:
gathering selection parameters representing neurologic problems in a patient;
verifying if the selection parameters is representing presence of neurologic problems;
in the case it is, transmitting a first response signal to the evaluation module, configured for managing the urodynamic-evaluation for the patient with neurologic problems, and for transmitting the signals representing the urodynamic-evaluation to the user interface;
otherwise, transmitting a second response signal to the determination module for managing the non-neurologic case.

15. The method according to claim 14 comprising the step of:
providing a visual interface for gathering parameters and/or for displaying the signals representing the urodynamic-evaluation.

16. The method according to claim 15 comprising the step of:
providing a network interface for gathering parameters and/or for displaying the signals representing the urodynamic-evaluation.
